(19) United States
(12) Patent Application Publication
(10) Pub. No.: US 2013/0136710 A1
(43) Pub. Date: May 30, 2013

(54) ANTIPERSPIRANT COMPOSITIONS HAVING STABLE VISCOSITY, ANTIPERSPIRANT PRODUCTS COMPRISING SUCH ANTIPERSPIRANT COMPOSITIONS, AND METHODS FOR MAKING SUCH ANTIPERSPIRANT COMPOSITIONS

(71) Applicant: The Dial Corporation, Scottsdale, AZ (US)

(72) Inventor: Haiyan Ge, Scottsdale, AZ (US)

(73) Assignee: The Dial Corporation, Scottsdale, AZ (US)

(21) Appl. No.: 13/713,185

(22) Filed: Dec. 13, 2012

Related U.S. Application Data
(63) Continuation-in-part of application No. 13/307,706, filed on Nov. 30, 2011.

(51) Int. Cl. A61K 8/19; A61Q 15/00

(52) U.S. Cl. CPC A61K 8/19; A61Q 15/00
USPC 424/65

(57) ABSTRACT

An antiperspirant composition includes an active antiperspirant component; a silicone carrier fluid; a mineral clay mixture treated with a quaternary ammonium compound; and a stabilizer. The mineral clay mixture includes (a) a mineral clay chosen from sepiolite, palygorskite, and combinations thereof; and (b) a smectite mineral clay. A ratio of the stabilizer to the clay mixture is from 6.5 to 11 weight %.

### TECHNICAL FIELD

The present invention relates generally to antiperspirant compositions, products comprising the antiperspirant compositions, and methods for making the antiperspirant compositions. More particularly, the present invention relates to antiperspirant compositions that comprise a suspension having stable viscosity, antiperspirant products comprising such antiperspirant compositions, and methods for making such antiperspirant compositions.

### BACKGROUND

Antiperspirant and deodorant compositions are well known personal care products used to prevent or eliminate perspiration and body odor caused by perspiration. The compositions come in a variety of forms and may be formulated, for example, into aerosols, pumps, sprays, liquids, roll-ons, lotions, creams, sticks, and soft solids, etc.

Various types of antiperspirant compositions are desirable by a large majority of the population because of their ease of application and the presence of active antiperspirant compounds (e.g., antiperspirant salts) that prevent or block the secretion of perspiration and its accompanying odors. One type of antiperspirant composition comprises a suspension of the antiperspirant compounds in a silicone carrier fluid along with other additives such as fragrances and structurants. Such antiperspirant compositions can be applied to the skin through roll-on application to the skin, typically of the underarm. Upon application, the carrier fluid evaporates, leaving the active antiperspirant compound from the antiperspirant composition on the skin.

Because active antiperspirant compounds settle out of the antiperspirant compositions over time, viscosity of the antiperspirant compositions is a consideration for purposes of establishing consistent antiperspirant efficacy and ease of application of the antiperspirant composition. To control viscosity of the antiperspirant compositions, structurants such as organoclays have been included in the antiperspirant compositions. Organoclays typically represent the reaction product of a smectite-type clay with a quaternary ammonium compound. However, many organoclays settle out of the antiperspirant compositions over time, especially when the antiperspirant compositions are stored at temperatures above room temperature, resulting in poor viscosity stability of the antiperspirant compositions. Once the organoclays settle out of the antiperspirant compositions, the original viscosity of the antiperspirant compositions often cannot be recaptured.

Accordingly, it is desirable to provide antiperspirant compositions that comprise a suspension of an active antiperspirant component in a silicone carrier fluid and that exhibit stable viscosity over time. In addition, it is desirable to provide antiperspirant products comprising such antiperspirant compositions. It is also desirable to provide methods for making the antiperspirant compositions. Furthermore, other desirable features and characteristics of the present invention will become apparent from the subsequent detailed description of the invention and the appended claims, taken in conjunction with the accompanying drawings and this background of the invention.

### BRIEF SUMMARY

An antiperspirant composition includes an active antiperspirant component; a silicone carrier fluid; a mineral clay mixture treated with a quaternary ammonium compound; and a stabilizer. The mineral clay mixture includes (a) a mineral clay chosen from sepiolite, palygorskite, and combinations thereof; and (b) a smectite mineral clay. A ratio of the stabilizer to the clay mixture is from 6.5 to 11 weight %.

An antiperspirant product includes a container; and an antiperspirant composition housed within the container. The antiperspirant composition includes an active antiperspirant component; a silicone carrier fluid; a mineral clay mixture treated with a quaternary ammonium compound; and a stabilizer. The mineral clay mixture includes (a) a mineral clay chosen from sepiolite, palygorskite, and combinations thereof; and (b) a smectite mineral clay. A ratio of the stabilizer to the clay mixture is from 6.5 to 11 weight %.

A method for making an antiperspirant composition includes mixing a silicone carrier fluid, an active antiperspirant component, a stabilizer and a mineral clay mixture to form the antiperspirant composition. The mineral clay mixture is treated with a quaternary ammonium compound and includes: (a) a mineral clay chosen from sepiolite, palygorskite, and combinations thereof; and (b) a smectite mineral clay. A ratio of the stabilizer to the mineral clay mixture is from 6.5 to 11 weight %.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

An antiperspirant composition, an antiperspirant product comprising the antiperspirant compositions, and a method of making the antiperspirant composition are provided herein. The antiperspirant composition is in the form of a suspension of an active antiperspirant component in a silicone carrier fluid, and is intended for application to skin of a consumer (typically of the underarm). Viscosity of the antiperspirant composition is, for example, within a range of from about 1650 to about 5000 Centipoise (cps), such as from about 2000 to about 3500 cps, as measured at 22° C., which is similar to viscosity of a light syrup. As such, the antiperspirant composition is typically best suited for application through spray, roll-on, or dab-on techniques. However, it is to be appreciated that the antiperspirant composition described herein may be applied through any application technique known in the art.

The active antiperspirant component is not particularly limited, but preferably comprises one or more active antiperspirant compounds that are effective when included in antiperspirant compositions in suspension form. In this regard, the “active antiperspirant component” represents all active antiperspirant compounds that are present in the antiperspirant composition. Active antiperspirant compounds are known in the art. Examples of preferred active antiperspirant compounds include astringent water-soluble inorganic and organic salts of aluminum, zirconium and zinc or any mixtures of these salts. In accordance with the instant application, water-solubility refers to a solubility of at least 5% by weight at 20° C. (i.e., at least 5 g of active antiperspirant compound is soluble in 95 g of water at 20° C.)

Some active antiperspirant compounds that may be used include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In an embodiment, the active antiperspirant compound is aluminum zirconium trichlorohydrex glycine complex. In another embodiment, the active antiperspirant compound is an aluminum-zirconium-pentachlorohydrex-glycine complex, which complexes for example are commercially available from Summit Reheis of Huguenot, N.Y. under the designations AAZG-3108 and AAZG-3110 as powders.

In an embodiment, at least one of the active antiperspirant compounds is provided in particulate or powder form. In this regard, the aforementioned activated aluminum and aluminum-zirconium salts may be dried into a powder, e.g., through spray or roller drying, before use in the antiperspirant composition. To enable formation of the suspension, the particulate or powder, for example, has an average nominal diameter of from about 0.1 to about 50 μm, such as from about 1 to about 20 μm. However, it is to be appreciated that non-powder active antiperspirant compounds may be included in the antiperspirant composition in addition to active antiperspirant compounds that are in particulate or powder form.

The antiperspirant component is present in the antiperspirant composition in an amount that provides a measurable antiperspirant effect when applied to the underarms of a consumer. In an embodiment, the active antiperspirant component is present in an amount of from about 10 to about 25 weight %, for example, from 15 to 25 weight %, based upon the total weight of the antiperspirant composition. It is to be appreciated that the above values represent the total amount of all active antiperspirant compounds present in the antiperspirant composition. It is also to be appreciated that most commercially available active antiperspirant compounds are sold in mixtures along with other compounds that are not considered active antiperspirant compounds, and the above values account for the presence of such non-active antiperspirant compounds. When sold in mixtures, the active antiperspirant composition includes antiperspirant active compounds in an amount of from about 30 to about 99 weight %, such as from about 50 to about 85 weight %, based on the total weight of the active antiperspirant component.

As set forth above, the antiperspirant composition also comprises a silicone carrier fluid. The “silicone carrier fluid” represents all liquid silicones that are present in the antiperspirant composition. Examples of suitable silicone carrier fluids include liquid siloxanes and, particularly, volatile polyorganosiloxanes having a measurable vapor pressure at ambient conditions. The polyorganosiloxanes can be linear or cyclic or combinations thereof. Exemplary siloxanes include cyclomethicones such as cyclotetrasiloxane, cyclopentasiloxane, cyclohexasiloxane, and combinations thereof. It is to be appreciated that the silicone carrier fluid may also comprise non-volatile silicones, which may be included for various skin-conditioning purposes. However, in an embodiment, such non-volatile silicones are preferably present in a total amount of less than or equal to 1% by weight based upon the total weight of the antiperspirant composition to avoid excessive residual wetness after the antiperspirant composition dries. The silicone carrier fluid is present within the antiperspirant composition in an amount sufficient to provide a continuous phase of the suspension. In an embodiment, the silicone carrier fluid is present in an amount of from about 60 to about 85 weight %, for example, from about 65 to about 80 weight %, based upon the total weight of the antiperspirant composition.

The antiperspirant composition further includes a mineral clay mixture. The mineral clay mixture that is included in the antiperspirant composition described herein enables the antiperspirant composition to maintain excellent viscosity stability over time, i.e., a relatively small change between the initial viscosity and the viscosity of the antiperspirant composition after storage for a period of time and after re-dispersion of separated layers in the antiperspirant composition in the event of settling, as described in further detail below. By maintaining excellent viscosity stability over time, the antiperspirant composition exhibits consistent antiperspirant efficacy, which may otherwise be compromised in antiperspirant compositions having poor viscosity stability. Furthermore, the excellent viscosity stability promotes ease of application of the antiperspirant composition even under conditions in which the antiperspirant composition has been stored.

The mineral clay mixture includes (a) a mineral clay chosen from sepiolite, palygorskite, and combinations thereof, and (b) a smectite mineral clay. As described in more detail below, the mineral clay mixture is treated with a quaternary ammonium compound. Without intending to be bound by any particular theory, it is believed that the mineral clay mixture, by including the mineral clay (a) which has a rod-type structure and mineral clay (b) which has a platelet-type structure, resists layer formation upon settling due to the non-uniformity that is introduced through the presence of rod-type and platelet-type structures of the respective mineral clays. In particular, it is believed that the non-uniformity that is introduced through the presence of rod-type and platelet-type structures enables formation of spaces between the platelets that enable particles of the active antiperspirant component to be suspended between the platelets. It is also believed that alignment of platelets upon settling (which may result when only platelet-type mineral clays are used due to alignment of the platelets) impacts viscosity stability by resisting re-dispersion after settling, thereby resulting in permanent loss of viscosity. In contrast, re-dispersion of settled mineral clay is possible in the antiperspirant compositions described herein that include the mineral clay mixture. The quaternary ammonium compound treatment of the mineral clay mixture also assists with dispersion of the mineral clay mixture by providing repulsive steric hindrance to the particles.

Sepiolite and palygorskite of the mineral clay (a) are papyrus-like or fibrous hydrated magnesium silicate minerals (i.e., rod-type) and are included in the phyllosilicate group of mineral clays because they contain a continuous two-dimensional tetrahedral sheet of composition Si₂O₅. They differ, however, from other layer silicates in the phyllosilicate group because they lack continuous octahedral sheets. The structures of sepiolite and palygorskite are alike and can be regarded as consisting of narrow strips or ribbons of 2:1 layers of tetrahedral and octahedral sheets that are linked stepwise at the corners. One ribbon is linked to the next by inversion of the direction of the apical oxygen atoms of SiO₄ tetrahedrons; in other words, an elongated rectangular box consisting of continuous 2:1 layers is attached to the nearest boxes at their elongated corner edges. Therefore, channels or tunnels due to the absence of the silicate layers occur on the elongated sides of the boxes. The elongation of the structural element is related to the fibrous morphology of the minerals and is parallel to the a axis. Since the octahedral sheet is discontinuous, some octahedral magnesium ions are exposed at the edges and hold bound water molecules (OH₂). In addition to the bound water, variable amounts of zeolitic (i.e., free) water (H₂O) are contained in the rectangular channels. The major difference between the structures of sepiolite and palygorskite is the width of the ribbons, which is greater in sepiolite than in palygorskite. The width determines the number of octahedral cation positions per formula unit. Thus, sepiolite and palygorskite have the ideal compositions Mg₈Si₁₂O₃₀(OH)₄(OH₂)₄(H₂O)₈ and (Mg, Al)₅Si₈O₂₀(OH)₂(OH₂)₄(H₂O)₄, respectively.

Smectite mineral clays include those in the general class of clay minerals with di- and tri-octahedral structures and have the platelet-type structure referred to above. Dioctahedral smectites include montmorillonite, beidelite, and nontronite; trioctahedral smectites includes saponite, hectorite, stevensite and sauconite. Also included are the synthetically prepared smectite clays. In this regard, the smectite mineral clay (b) may be, for example, hectorite, montmorillonite, bentonite, beidelite, saponite, stevensite, nontronite, sauconite, and combinations thereof.

In regards to relevant amounts of the mineral clay (a) and mineral clay (b) in the mineral clay mixture, in an embodiment, the mineral clay (a) is present in an amount of from about 50 to about 95 weight %, for example from about 70 to about 90 weight %, based on the total weight of the mineral clay mixture, with a balance of the mineral clay mixture being the mineral clay (b).

As set forth above, the mineral clay mixture is treated with a quaternary ammonium compound. Suitable quaternary ammonium compounds for treating mineral clays, as well as methods of treating mineral clays with such compounds, are known in the art. Suitable quaternary ammonium compounds include alkyl quaternary ammonium salts containing the same or different straight- and/or branched-chain saturated and/or unsaturated alkyl groups of 1 to 22 carbon atoms. The salt moiety may be a chloride, bromide, methyl sulfate, nitrate, hydroxide, acetate, phosphate and combinations thereof, preferably chloride, bromide and methylsulfate. Exemplary alkyl quaternary ammonium salts include dimethyl di(hydrogenated tallow) ammonium chloride, methylbenzyl di(hydrogenated tallow) ammonium chloride, dimethylbenzyl hydrogenated tallow ammonium chloride, dimethyl hydrogenated tallow-2-ethylhexylammonium methylsulfate and mixtures of two or more of the exemplary salts. The mineral clay mixture is typically treated with 5 to 80 milliequivalent (meq.) of the quaternary ammonium compound per 100 g of the mixture.

In an embodiment of a procedure for preparing the mineral clay mixture, the sepiolite and/or palygorskite mineral clay (a) is crushed, ground, slurried in water and screened to remove grit and other impurities. The smectite mineral clay (b) is subjected to a similar regimen. Each of the slurries of mineral clays (a) and (b) is then subjected as a dilute (1 to 6% solids) aqueous slurry to high shearing in a suitable mill. For use in this shearing step, a homogenizing mill may be used of the type wherein high speed fluid shear of the slurry is effected by passing the slurry at high velocities through a narrow gap, across which a high pressure differential is maintained. This type of action can be effected in the well-known Manton-Gaulin “MG” mill, which device is sometimes referred to as the “Gaulin homogenizer”. The conditions for use of the MG mill are known in the art. The slurry to be treated may be passed one or more times through the MG mill. Among additional instrumentalities which can be effectively utilized to provide high shearing of the clay components, is the rotor and stator arrangement described in U.S. Pat. No. 5,160,454.

Following the high shear step, the slurries of mineral clays (a) and (b) may be mixed with one another. Alternatively, the slurries of mineral clays (a) and (b) can be intermixed in a single slurry before the latter is subjected to the high shear step. Following such step the single slurry is intermixed with the quaternary ammonium compound, after which the slurry is dewatered, and the quaternary ammonium-treated mineral clay mixture is dried and ground to provide a dry mineral clay mixture.

Mineral clay mixtures containing mineral clays (a) and (b) that have been treated with quaternary ammonium compounds are commercially available. Specific examples of commercially available mineral clay mixtures include, but are not limited to, those sold under the name Garamite® by Southern Clay Products, Inc. of Gonzales, Tex.

In an embodiment, the mineral clay mixture is preferably present in the antiperspirant composition in an amount of from about 2.0 to about 5.2 weight %, for example, from about 2.7 to about 3.7 weight %, such as from about 2.9 to about 3.3 weight %. The amount of the mineral clay mixture in the antiperspirant composition may be selected based upon the particular viscosity desired for the antiperspirant composition.

In an embodiment, the antiperspirant composition is preferably free of stabilizer or stabilizers. For purposes of the instant application, “free of stabilizer(s)” means that stabilizer(s) are either absent from the antiperspirant composition altogether, or are present in an amount so low that the stabilizer(s) have no effect on any functional aspect of the antiperspirant composition. In one embodiment, “free of stabilizer(s)” means less than 0.02 wt. %.

In an alternative embodiment, however, the antiperspirant composition may include amounts of a stabilizer or stabilizers, also known in the art as activators, such as propylene carbonate, ethanol and mixtures thereof. Specifically, in compositions that include a mineral clay mixture including a mineral clay (a) which has a rod-type structure and mineral clay (b) which has a platelet-type structure, a ratio of stabilizer to clay ranges from 6.5 to 11 weight %. As above, these compositions include a mixture of (a) a mineral clay chosen from sepiolite, palygorskite, and combinations thereof, and (b) a smectite mineral clay.

This range of stabilizer-to-clay ratios is substantially below the quantities of stabilizer traditionally used in antiperspirant compositions. As noted above, antiperspirant compositions with a mineral clay mixture of a rod-type structure clay and a platelet-type structure clay can be stable even if free of stabilizer. However, it has been further discovered that the introduction of reduced amounts of stabilizer with the range of ratios noted above provides unexpected and significant benefits.

In addition to the aforementioned components, further additives may be included in the antiperspirant composition for various purposes including additives that cause the antiperspirant composition to exhibit long-lasting fragrance, odor protection, bacteria control, and/or another desired purpose and/or function. Specific examples of additional such additives include, but are not limited to, fragrances, including encapsulated fragrances; skin conditioners; dyes; pigments; preservatives; antioxidants; moisturizers; and the like. These optional additives can be included in the antiperspirant composition in a total amount of from about 0 to about 20 weight %. Fragrance, for example, may be present in an amount of from about 0.1 to about 0.5 weight %, such as, from about 0.2 to about 0.3 weight %, based on the total weight of the antiperspirant composition. For some of the optional additives, such as fragrances, viscosity of the antiperspirant composition may be impacted and it is to be appreciated that amounts of the mineral clay mixture in the antiperspirant composition may be adjusted to account for any effect of the optional additives on viscosity of the antiperspirant composition so as to maintain the viscosity of the antiperspirant composition within a desired range.

In one specific embodiment, the antiperspirant composition includes the active antiperspirant component present in an amount of from about 10 to about 25 weight %, the silicone carrier fluid present in an amount of from about 65 to about 85 weight %, and the mineral clay mixture present in an amount of from about 2.0 to about 5.2 weight %, all based on the total weight of said antiperspirant composition. Additionally, fragrance may be present in the antiperspirant composition in an amount of from about 0.2 to 0.3 weight % based upon the total weight of the antiperspirant composition in this embodiment.

As set forth above, the antiperspirant composition including the mineral clay mixture described herein exhibits excellent viscosity stability over time. As also set forth above, the antiperspirant composition preferably has a viscosity of from about 1650 to about 5000 cps at 22° C. Viscosity stability of the antiperspirant composition is preferably such that the antiperspirant composition has an initial viscosity of from about 1650 to about 5000 cps at 22° C. and a second viscosity of from about 1650 to about 5000 cps at 22° C. after maintaining the antiperspirant composition at a temperature of 40° C. for 2 months. Because the antiperspirant composition may exhibit non-uniform viscosity immediately after mixing the components therefor, initial viscosity for purposes of measuring viscosity stability is determined 10 hours after making the antiperspirant composition. The second viscosity is determined after mixing the antiperspirant composition, with the antiperspirant composition visibly appearing homogeneous with no apparent separation. Within the aforementioned range of viscosity of from about 1650 to about 5000 cps, the antiperspirant composition preferably exhibits a change in viscosity (between the initial viscosity and the second viscosity) of less than or equal to about 15% of the initial viscosity of the antiperspirant composition after maintaining the antiperspirant composition at a temperature of 40° C. for 2 months. In particular, by including the mineral clay mixture described herein, the viscosity of the antiperspirant composition can maintain stable viscosity even when the antiperspirant composition is stored at temperatures above room temperature.

The antiperspirant composition can be made through known techniques, and it is to be appreciated that the antiperspirant composition is not limited to any particular method of making the same. In one embodiment, the method for making the antiperspirant composition includes mixing the aforementioned silicone carrier fluid, the active antiperspirant component, and the mineral clay mixture to form the antiperspirant composition. Preferably, in one specific embodiment, the method for making an antiperspirant composition includes first mixing the aforementioned silicone carrier fluid and mineral clay mixture that has been treated with the quaternary ammonium compound to form a premix. Once the premix has been formed, the active antiperspirant component and the premix are mixed to form the antiperspirant composition. In this regard, adequate mixing of the mineral clay mixture and the silicone carrier fluid can be guaranteed prior to introduction of the active antiperspirant component. In this embodiment of the method, the premix preferably includes an entire amount of the silicone carrier fluid in the antiperspirant composition, which differs from conventional methods in which only a portion of the total silicone carrier fluid in the antiperspirant composition is included in the premix. By providing the entire amount of the silicone carrier fluid in the antiperspirant composition with the premix, viscosity of the resulting antiperspirant composition may beneficially be increased as compared to scenarios in which less than the entire amount of the silicone carrier fluid is provided within the premix. Shear time over which the antiperspirant composition is mixed may also affect viscosity of the resulting antiperspirant composition, although particular shear times and the effect on viscosity of the antiperspirant composition are highly dependent upon batch size among other factors.

As alluded to above, the antiperspirant composition is typically best suited for application through spray, roll-on, or dab-on techniques. In this regard, the antiperspirant composition may be provided to consumers, for example, in the form of the antiperspirant product that includes a container and the antiperspirant composition housed within the container. In one specific embodiment, the container is a roll-on container and may include a roller ball for enabling application of the antiperspirant composition to the skin. However, it is to be appreciated that the container may be suited for any of the aforementioned application techniques, and such containers are known in the art.

The following Examples are to be read as illustrative of the antiperspirant composition and method of making the same as described herein, and are not to be interpreted as limiting.

### EXAMPLES

The formulation of illustrative Antiperspirant Compositions A and B will now be described. These compositions can be prepared without adding a stabilizer, such as propylene carbonate. However, as will be detailed below, there are advantages to adding a stabilizer in certain limited quantities.

Antiperspirant Compositions A and B are prepared by mixing a silicone carrier fluid and mineral clay mixture that has been treated with the quaternary ammonium compound to form a premix. Once the premix has been formed, the active antiperspirant component and the premix are mixed to form the antiperspirant composition. For Antiperspirant Composition A, the premix includes 76.6 weight % of the silicone carrier fluid based upon the total weight of the final antiperspirant composition, which represents the entire amount of silicone carrier fluid included in the antiperspirant composition, while Antiperspirant Composition B includes 40 weight % of the silicone carrier fluid based upon the total weight of the final antiperspirant composition, which represents only a portion of the amount of silicone carrier fluid included in the antiperspirant composition. For Antiperspirant Composition B, the remaining silicone carrier fluid is post-added along with the active antiperspirant component and premix to form the resulting Antiperspirant Composition B. Identical mixing conditions and equipment were used to make Antiperspirant Compositions A and B, the only difference being the amount of silicone carrier fluid included in the premix for the respective antiperspirant compositions. Table I below provides the types and amounts of each component included within the antiperspirant compositions, with all amounts in weight % based upon the total weight of the antiperspirant compositions. Table I also provides initial viscosity of the antiperspirant compositions as measured 10 hours after making the antiperspirant compositions.

TABLE I

| | Antipers. Comp. A | Antipers. Comp. B |
|---|---|---|
| Cyclotetrasiloxane, Premix | 38.30 | 20.00 |
| Cyclopentasiloxane, Premix | 38.30 | 20.00 |
| Mineral Clay Mixture A | 3.10 | 3.10 |
| Active Antiperspirant Component | 20.00 | 20.00 |
| Cyclotetrasiloxane, Post Addition | 0.00 | 18.30 |
| Cyclopentasiloxane, Post Addition | 0.00 | 18.30 |
| Tocopheral acetate | 0.10 | 0.10 |
| Isopropyl myristate and | 0.10 | 0.10 |
| Gossypium Herbaceum seed oil | | |
| Fragrance | 0.10 | 0.10 |
| Total, weight % | 100.00 | 100.00 |
| Viscosity, cps @ 22° C. | ~3250 | ~2450 |

Mineral Clay Mixture A is Garamite® 1958V, a clay mixture of 50-95 wt. % sepiolite, palygorskite or a mixture of the two, with the balance of the clay mixture being smectite, that has been treated with a quaternary ammonium compound, commercially available from Southern Clay Products, Inc.

Active Antiperspirant Component includes 81.5 weight % aluminum zirconium pentachlorohydrex, with the balance being glycine and water.

In addition, a stabilizer is added to either composition A or B, where the stabilizer is propylene carbonate, ethanol and mixtures of the two. The stabilizer is added in a quantity such that the ratio of stabilizer-to-clay ranges from, for example, 6.5 to 11 weight %.

In another set of experiments, shear time for mixing the premix that is then used to form Antiperspirant Compositions A and B was varied, with the temperature range at which mixing was performed also varying. Temperature of the premix that was sheared for 4 minutes was about 25° C. after shearing, while temperature of the premix that was sheared for 12 minutes was about 36° C. after shearing. The pre-mix was first prepared in accordance with the specified shear times, followed by adding the active antiperspirant component, and then adding any remaining silicone carrier fluid. The differences in temperature of the premix also resulted in corresponding differences of the antiperspirant compositions after post-addition of the additional components. Total batch size was about 2500 g, and an Arde-Barinco mixer, model CJ4C was used. Table II provides initial viscosity of the antiperspirant compositions as measured 10 hours after making the antiperspirant compositions.

TABLE II

| | Antipers. | Antipers. | Antipers. | Antipers. |
|---|---|---|---|---|
| | Comp. A | Comp. B | Comp. A | Comp. B |
| Shear Time of | X | X | | |
| Premix, 4 min. | | | | |
| Shear Time of | | | X | X |
| Premix, 12 min. | | | | |
| Viscosity of | ~3200 | ~2500 | ~2500 | ~2000 |
| Antiperspirant | | | | |
| Composition, cps | | | | |
| @ 22° C. | | | | |

Antiperspirant Compositions C-G were prepared in an identical manner with no premixing step and with varying amounts of the Mineral Clay Mixture A (which affects the amount of each component within the antiperspirant compositions as a percentage thereof). Table III below provides the types and amounts of each component included within the antiperspirant compositions, with all amounts in weight % based upon the total weight of the antiperspirant compositions, and also provides initial viscosity of the antiperspirant compositions as measured 10 hours after making the antiperspirant compositions.

TABLE III

| | Antipers. | Antipers. | Antipers. | Antipers. | Antipers. |
|---|---|---|---|---|---|
| | Comp. C | Comp. D | Comp. E | Comp. F | Comp. G |
| Cyclotetrasiloxane | 38.50 | 38.45 | 38.40 | 38.30 | 37.85 |
| Cyclopentasiloxane | 38.50 | 38.45 | 38.40 | 38.30 | 37.85 |
| Mineral Clay Mixture | 2.70 | 2.80 | 2.90 | 3.10 | 4.00 |
| Active Antiperspirant | 20.00 | 20.00 | 20.00 | 20.00 | 20.00 |
| Component | | | | | |
| Tocopheral acetate | 0.10 | 0.10 | 0.10 | 0.10 | 0.10 |
| Isopropyl myristate and | 0.10 | 0.10 | 0.10 | 0.10 | 0.10 |
| Gossypium Herbaceum seed oil | | | | | |
| Fragrance | 0.10 | 0.10 | 0.10 | 0.10 | 0.10 |
| Total, weight % | 100.00 | 100.00 | 100.00 | 100.00 | 100.00 |
| Viscosity, cps @ 22° C. | ~2600 | ~2950 | ~3000 | ~3180 | ~3800 |

Viscosity stability after storage for 2 months was tested for Antiperspirant Composition F at various storage temperatures, with the antiperspirant compositions stored in sealed containers and with settled phases of the antiperspirant compositions re-distributed through vigorous shaking of the containers by hand until visually homogeneous. Table IV provides the initial (as measured 10 hours after making the antiperspirant compositions) and second viscosities of the antiperspirant compositions.

TABLE IV

| | Initial | 1 Month | 2 Month | 3 Month |
|---|---|---|---|---|
| | Viscosity, | Viscosity, | Viscosity, | Viscosity, |
| | cps @ 22° C. | cps @ 22° C. | cps @ 22° C. | cps @ 22° C. |
| Storage Temp., | 3180 | 3480 | 3520 | 3530 |
| 4° C. | | | | |
| Storage Temp., | 3180 | 3420 | 3400 | 3360 |
| 25° C. | | | | |
| Storage Temp., | 3180 | 3470 | 3200 | 3920 |
| 40° C. | | | | |
| Storage Temp., | 3180 | 3550 | 3000 | — |
| 45° C. | | | | |

### Example 1

This example uses Garamite® 1958V from Southern Clay Products, Inc., which is a mixture of a rod-type structure clay and a platelet-type structure clay. The chemical name for Garamite® 1958V is Alkyl Quaternary Ammonium Clay. In this formulation, propylene carbonate is added as a stabilizer in a stabilizer-to-clay ratio of 7.5 to 11 weight %. More specifically, the stabilizer-to-clay ratio may be 8 to 9 weight %. The weight percentage of stabilizer in the formulation may range from 0.119 to 0.33 weight %.

Example 1 has been shown to provide stable viscosity over time at storage temperatures ranging from 4 to 45° C. Additionally, the formulation of Example 1 had less white residue that might transfer to the clothing of a user than formulations described herein lacking an added stabilizer. The formulations of Example 1 were also shown to have a drier feel among a panel of test users as compared to formulations that contains Bentone 38 VCG clay and stabilizer with a stabilizer-to-clay ratio of about 30 weight %.

One specific formulation of the present example is detailed in Table V below.

TABLE V

| | | Raw | | |
|---|---|---|---|---|
| | | Material | % | % |
| Ingredient | Function | Purity (%) | Active | Weight |
| DC-245 | Carrier | 100 | 38.765 | 38.7650 |
| (Cyclopentasiloxane) | fluid | | | |
| DC-244 | Carrier | 100 | 38.765 | 38.7650 |
| (Octamethylcyclotetra- | fluid | | | |
| siloxane) | | | | |
| Garamite ® 1958V | Structrant | 100 | 2.000 | 2.0000 |
| Propylene carbonate | Stabilizer | 100 | 0.168 | 0.168 |
| Summit ™ AAZG- | Active | 81.5 | 16.300 | 20.0000 |
| 3108 (Aluminum | | | | |
| zirconium | | | | |
| Pentachlorohydrex | | | | |
| glycine complex) | | | | |
| Tocopheryl Acetate | Skin | 100 | 0.100 | 0.1000 |
| (Vitamin E Acetate) | conditioner | | | |
| Isopropyl Myristate | Skin | 100 | 0.100 | 0.1000 |
| & Gossypium | conditioner | | | |
| Herbaceum (Cotton) | | | | |
| Seed Oil | | | | |
| Fragrance | Fragrance | 100 | 0.100 | 0.1000 |
| | | TOTALS: | 96.30 | 100.00 |

The carrier fluids listed, i.e., DC-245 and DC-244 are available from Dow Corning®. DC-244 is primarily octamethylcyclotetrasiloxane, but may include some amount, for example less than or equal to 5 weight %, of hexamethyl cyclotrisiloxane in addition to octamethylcyclotetrasiloxane The Aluminum zirconium Pentachlorohydrex glycine complex in the form of Summit™ AAZG-3108 is a product of SummitReheis, Inc. of Huguenot, N.Y.

Referring to Table V above, the first three listed ingredients, i.e., the carrier fluids and the mineral clay mixture, are added together and sheared. For example, the shearing was performed for 25 minutes at a temperature of 30° C. Shearing may be performed, for example, with an Arde-Barinco mixer, model CJ4C

Then, the stabilizer, propylene carbonate, was added and the mixture was again sheared, for example, for 3 minutes. Finally, the rest of the ingredients were added and the mixture was again sheared, for example, for 3 minutes.

### Example 2

This example uses Garamite® 7303, also from Southern Clay Products, Inc., which is a mixture of a rod-type structure clay and a platelet-type structure clay. The International Nomenclature of Cosmetic Ingredients (INCI) name for Garamite® 7303 is Quaternium-90 Sepiolite and Quaternium-90 Montmorillonite. As compared with Garamite® 1958V, Garamite® 7303 is prepared using a single surfactant, i.e., Quaternium-90 Sepiolite.

In this formulation, propylene carbonate is added as a stabilizer in a stabilizer-to-clay ratio of 6.5 to 9.5 weight %. In one specific example, the stabilizer-to-clay ratio may be 6.81 weight %. The weight percentage of stabilizer in the formulation may range from 0.104 to 0.285 weight %.

Example 2 has been shown to provide stable viscosity over time at storage temperatures ranging from 4 to 45° C. Additionally, the formulation of Example 2 had less white residue that might transfer to the clothing of a user that formulations described herein lacking an added stabilizer.

One specific formulation of the present example is detailed in Table VI below.

TABLE VI

| | | Raw | | |
|---|---|---|---|---|
| | | Material | % | % |
| Ingredient | Function | Purity (%) | Active | Weight |
| DC-245 | Carrier fluid | 100 | 38.8887 | 38.8887 |
| (Cyclopentasiloxane) | | | | |
| DC-244 | Carrier fluid | 100 | 38.8887 | 38.8887 |
| (Octamethylcyclotetra- | | | | |
| siloxane) | | | | |
| Garamite ® 7303 | Structrant | 100 | 1.8000 | 1.8000 |
| Propylene carbonate | Stabilizer | 100 | 0.1226 | 0.1226 |
| Summit AAZG-3108 | Active | 81.5 | 16.3000 | 20.0000 |
| (Aluminum | | | | |
| zirconium | | | | |
| Pentachlorohydrex | | | | |
| glycine complex) | | | | |
| Tocopheryl Acetate | Skin conditioner | 100 | 0.1000 | 0.1000 |
| (Vitamin E Acetate) | | | | |
| Isopropyl Myristate | Skin conditioner | 100 | 0.1000 | 0.1000 |
| & Gossypium | | | | |
| Herbaceum (Cotton) | | | | |
| Seed Oil | | | | |
| Fragrance | Fragrance | 100 | 0.1000 | 0.1000 |
| | | TOTALS: | 96.30 | 100.00 |

Referring to Table VI above, the first three listed ingredients, i.e., the carrier fluids and the mineral clay mixture, are added together and sheared. For example, the shearing was performed for 25 minutes at a temperature of 30° C. Then, the stabilizer, propylene carbonate, was added and the mixture was again sheared, for example, for 3 minutes. Finally, the rest of the ingredients were added and the mixture was again sheared, for example, for 3 minutes.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. An antiperspirant composition comprising:
an active antiperspirant component;
a silicone carrier fluid;
a mineral clay mixture treated with a quaternary ammonium compound, said mineral clay mixture comprising:
(a) a mineral clay chosen from sepiolite, palygorskite, and combinations thereof; and
(b) a smectite mineral clay; and
a stabilizer, wherein a ratio of the stabilizer to the clay mixture is from 6.5 to 11 weight %.

2. The antiperspirant composition of claim 1, wherein said stabilizer comprises propylene carbonate.

3. The antiperspirant composition of claim 2, wherein said stabilizer comprises ethanol.

4. The antiperspirant composition of claim 1, wherein said stabilizer comprises ethanol.

5. The antiperspirant composition of claim 1, wherein said mineral clay mixture comprises Quaternium-90 Sepiolite and Quaternium-90 Montmorillonite, and said stabilizer is included in a ratio of stabilizer-to-clay of 6.5 to 9.5 weight %.

6. The antiperspirant composition of claim 5, wherein said stabilizer is included in a ratio of stabilizer-to-clay of 6.81 weight %.

7. The antiperspirant composition of claim 1, wherein said mineral clay mixture comprises Alkyl Quaternary Ammonium Clay, and said stabilizer is included in a ratio of stabilizer-to-clay of 7.5 to 11.0 weight %.

8. The antiperspirant composition of claim 7, wherein said stabilizer is included in a ratio of stabilizer-to-clay of 8 to 9 weight %.

9. The antiperspirant composition of claim 1 wherein said smectite mineral clay (b) is chosen from of hectorite, montmorillonite, bentonite, beidelite, saponite, stevensite, nontronite, sauconite, and combinations thereof.

10. The antiperspirant composition of claim 1, further comprising a skin conditioner.

11. The antiperspirant composition of claim 10, wherein said skin conditioner comprises one of Vitamin E Acetate, Cotton Seed Oil, and a mixture thereof.

12. The antiperspirant composition of claim 1, further comprising a fragrance component.

13. An antiperspirant product comprising:
a container; and
an antiperspirant composition housed within the container and comprising:
an active antiperspirant component;
a silicone carrier fluid;
a mineral clay mixture treated with a quaternary ammonium compound, said mineral clay mixture comprising:
(a) a mineral clay chosen from sepiolite, palygorskite, and combinations thereof; and
(b) a smectite mineral clay; and
a stabilizer, wherein a ratio of the stabilizer to the clay mixture is from 6.5 to 11 weight %.

14. The antiperspirant product of claim 13, wherein said container is further defined as a roll-on container.

15. The antiperspirant product of claim 13, wherein said stabilizer comprises propylene carbonate, ethanol or a combination thereof.

16. The antiperspirant product of claim 13, wherein said mineral clay mixture comprises Quaternium-90 Sepiolite and Quaternium-90 Montmorillonite, and said stabilizer is included in a ratio of stabilizer-to-clay of 6.5 to 9.5 weight %.

17. The antiperspirant product of claim 16, wherein said stabilizer is included in a ratio of stabilizer-to-clay of 6.81 weight %.

18. The antiperspirant product of claim 13, wherein said mineral clay mixture comprises Alkyl Quaternary Ammonium Clay, and said stabilizer is included in a ratio of stabilizer-to-clay of 7.5 to 11.0 weight %.

19. The antiperspirant product of claim 18, wherein said stabilizer is included in a ratio of stabilizer-to-clay of 8 to 9 weight %.

20. A method for making an antiperspirant composition, the method comprising the steps of:
mixing a silicone carrier fluid, an active antiperspirant component, a stabilizer and a mineral clay mixture to form the antiperspirant composition, the mineral clay mixture treated with a quaternary ammonium compound and comprising:
(a) a mineral clay chosen from sepiolite, palygorskite, and combinations thereof; and
(b) a smectite mineral clay;
wherein a ratio of the stabilizer to the mineral clay mixture is from 6.5 to 11 weight %.
